(19) United States
(12) Patent Application Publication
(10) Pub. No.: US 2024/0350254 A1
(43) Pub. Date: Oct. 24, 2024

(54) SYSTEMS FOR COMPUTER-ASSISTED VERIFICATION, AND POSITIONING AND POSITION ANALYSIS OF INTRAOCULAR LENSES

(71) Applicant: Carl Zeiss Meditec AG, Jena (DE)

(72) Inventors: Jennifer-Magdalena Masch, Ratzeburg (DE); Karsten Laubner, München (DE); Thomas Lang, Oberhaching (DE); Benjamin Schreiber, Berlin (DE); Thorben Badur, Berlin (DE)

(73) Assignee: Carl Zeiss Meditec AG, Jena (DE)

(21) Appl. No.: 18/685,974

(22) PCT Filed: Aug. 24, 2022
(86) PCT No.: PCT/EP2022/073632

§ 371 Date: Feb. 23, 2024

(30) Foreign Application Priority Data
Aug. 25, 2021 (DE) 10 2021 209 352.3
Oct. 26, 2021 (DE) 10 2021 212 091.1

(51) Int. Cl. A61F 2/16; A61B 34/20; A61B 90/00; A61B 90/96; A61B 90/98

(52) U.S. Cl. CPC A61F 2/1613; A61B 34/20; A61B 90/96; A61B 2034/2065; A61B 2090/3735; A61B 2090/3904; A61B 90/98; A61F 2250/0096

(57) ABSTRACT

An ophthalmic implant has at least one marker element, wherein the at least one marker element is designed to provide information for the machine-based identification and/or characterisation of the ophthalmic implant and information for the machine-based determination of an orientation of the ophthalmic implant. The marker element is arranged in and/or on the ophthalmic implant such that the information for identifying and/or characterising the ophthalmic implant and/or the information for the machine-based determination of the orientation of the ophthalmic implant is intraoperatively and/or postoperatively machine-readable.

### CROSS REFERENCE TO RELATED APPLICATIONS

This application is a national stage application of International patent application PCT/EP2022/073632, filed on Aug. 24, 2022, designating the U.S. and claiming priority from German patent applications DE 10 2021 209 352.3, filed on Aug. 25, 2021, and DE 10 2021 212 091.1, filed on Oct. 26, 2021, and the entire content of all applications is incorporated herein by reference.

### TECHNICAL FIELD

The disclosure provides an ophthalmic implant, a data storage unit for an ophthalmic implant, the use of a marker element of an ophthalmic implant, a method for the machine-based identification of an ophthalmic implant and/or determination of an alignment and/or positioning of an ophthalmic implant, a planning apparatus, a readout apparatus, a system for providing machine-based assistance with the implantation of an ophthalmic implant, a method for manufacturing an intraocular lens, a computer program product, a computer-readable storage medium and a method for determining an eye position and/or eye movement. The exemplary embodiments are thus in particular in the field of ophthalmic implants, in particular intraocular lenses.

### BACKGROUND

In conventional cataract surgery, the pathologically altered, generally clouded, natural lens is extracted from the capsular bag by a cut in the cornea and replaced with an intraocular lens (IOL), which is introduced into the eye via the same cut and inserted into the capsular bag. In recent decades, cataract surgery has evolved from an implantation procedure that only replaces a clouded lens to a refractive procedure in which the implant both replaces the clouded natural lens and corrects any refractive error. This created a need for innovation with the aim of achieving the best possible postoperative visual results. For the best visual result, the IOL should be aligned in the optimum position in the eye, that is to say be aligned in a manner perfectly centered, not tilted and so as to be torically precise, and should have the correct depth position in the capsular bag.

An IOL is positioned and aligned in the capsular bag by the surgeon, and the positioning and alignment may be assisted by computer-aided surgical systems such as Zeiss CALLISTO eye, Alcon ORA or Alcon VERION, or intraoperative imaging such as OCT, in order to provide the surgeon with intraoperative guidance. These systems offer different functionalities specifically for the alignment of toric IOLs. A toric IOL is a lens that has two different refractive indices in two mutually perpendicular directions. Intraoperative OCT is able to provide visual monitoring of the IOL position, and intraoperative wavefront aberrometry is able to check the refractive result.

However, the success of the precise positioning and/or alignment of the IOL is based mainly on the surgeon's ability and skill, since conventional surgical systems offer no or at most very rudimentary support in this regard. As a rule, the surgical systems are a “black box” to the surgeon, which is accompanied by limited repeatability of the achieved precision. The reasons for the limited repeatability are in particular only implicit information about the IOL position provided by refractive wavefront measurements, limited precision due to varying surgical parameters and variations due to the patient's eye being in an abnormal/unnatural state, and/or inefficiency due to multiple measurements.

Conventional computer-aided surgical systems are not designed to reliably recognize the IOL position. Corresponding markers on IOLs currently only exist for toric IOLs. The surgeon is able to use these traditional markings to recognize the toric axis of the IOL, which the surgeon then has to align manually with the planned toric target axis. In many cases, the visibility of the markers is impaired by the operating conditions or the absolute position of the IOL in the eye during the implantation process. In this regard, current markers are optimized only so as to be visible or recognizable by the surgeon and offer the option of adjusting the axis position of the toric axis.

### SUMMARY

It is an object of the disclosure to facilitate the reliable implantation of an ophthalmic implant. The object may in particular comprise enabling precise, reproducible and efficient IOL positioning across all skill levels of the surgeon (minimum learning curve).

The object is achieved by an ophthalmic implant, a data storage unit for an ophthalmic implant, the use of a marker element of an ophthalmic implant, a method for the machine-based determination of an alignment and/or positioning of an ophthalmic implant, a planning apparatus, a readout apparatus, a system for providing machine-based assistance with the implantation of an ophthalmic implant, a method for manufacturing an intraocular lens, a computer program product, a computer-readable storage medium and a method for determining an eye position and/or eye movement, having the features set forth in the description.

A first exemplary embodiment relates to an ophthalmic implant having at least one marker element, wherein the at least one marker element is designed to provide information for the machine-based identification and/or characterization of the ophthalmic implant and information for the machine-based determination of an alignment of the ophthalmic implant. The marker element is furthermore arranged in and/or on the ophthalmic implant such that the information for the identification and/or characterization of the ophthalmic implant and the information for the machine-based determination of the alignment of the ophthalmic implant is able to be read out in a machine-based manner intraoperatively and/or postoperatively, that is to say in particular able to be read out in a machine-based manner in a state in which the ophthalmic implant is implanted in a patient's eye.

A further exemplary embodiment relates to a data storage unit for an ophthalmic implant, wherein the data storage unit is designed to store information for the machine-based identification and/or characterization of the ophthalmic implant in the form of electronically readable data. The data storage unit has such a spatial configuration and is able to be installed in and/or on the ophthalmic implant such that the alignment of the ophthalmic implant is able to be determined based on a position and/or alignment of the data storage unit in and/or on the ophthalmic implant.

A further exemplary embodiment relates to the use of a marker element of an ophthalmic implant or for an ophthalmic implant for providing information for the machine-based identification and/or characterization of the ophthalmic implant and for providing information for the machine-based determination of an alignment of the ophthalmic implant.

A further exemplary embodiment relates to the use of data storage element for an ophthalmic implant for providing information for the machine-based identification and/or characterization of the ophthalmic implant and for providing information for the machine-based determination of an alignment of the ophthalmic implant.

A further exemplary embodiment relates to a method for the machine-based determination of an alignment of an ophthalmic implant with at least one marker element relative to a patient's eye in order to assist an implantation operation. The method comprises machine-based, optionally optical and/or electronic, detection of the marker element. The method additionally comprises machine-based determination of a position and/or alignment of the marker element and machine-based determination of a position and/or an alignment of at least part of the patient's eye. The method additionally comprises determining a position and/or alignment of the ophthalmic implant relative to the patient's eye based on the determined position and/or alignment of the marker element and the determined position and/or alignment the at least part of the patient's eye, and also outputting information about the position and/or alignment of the ophthalmic implant relative to the patient's eye to a surgeon during the implantation operation.

A further exemplary embodiment relates to a system for exchanging information with an ophthalmic implant. The system comprises a write and/or readout apparatus that is designed to read out information for the machine-based identification and/or characterization of the ophthalmic implant from a marker element arranged in and/or on the ophthalmic implant or to write said information to the marker element. The write and/or readout apparatus is configured to optically detect the marker element in order to read out the information and/or to contactlessly establish an electrical communication connection with the ophthalmic implant. The system furthermore has a computing unit that is designed, based on the read-out information, to take an action concerning the ophthalmic implant and/or to output a notification to a user and/or, based on actions taken and/or to be taken and/or based on information provided by a user input, to provide the information for the machine-based identification and/or characterization of the ophthalmic implant to be written to the marker element, that is to say for example to plan a surgical procedure.

A further exemplary embodiment relates to a readout apparatus for reading out information for the machine-based identification and/or characterization of the ophthalmic implant from a marker element arranged in and/or on the ophthalmic implant, wherein the readout apparatus is configured to optically detect the marker element in order to read out the information and/or to contactlessly establish an electrical communication connection with the ophthalmic implant.

A further exemplary embodiment relates to a system for providing machine-based assistance for a user when implanting an ophthalmic implant in a patient's eye. The system comprises an image acquisition device for acquiring images of at least part of the patient's eye and of at least one marker element of the ophthalmic implant, and a computing unit that is configured to determine a relative position and/or alignment of the ophthalmic implant relative to the patient's eye based on the acquired images of the patient's eye and of the marker element. The system furthermore comprises an output unit that is configured to output information about the determined alignment of the ophthalmic implant relative to the patient's eye to a user.

A further exemplary embodiment relates to a method for manufacturing an intraocular lens. The method comprises providing an intraocular lens having at least one optical part and at least one haptic element connected to the optical part. The method furthermore comprises arranging a marker element containing information for the machine-based identification and/or characterization of the ophthalmic implant at least partially in and/or on the optical part and/or in and/or on the at least one haptic element such that, in a state in which the ophthalmic implant is implanted in a patient's eye, the information for the identification and/or characterization of the ophthalmic implant is able to be read out in a machine-based manner and information for the machine-based determination of the alignment of the ophthalmic implant is able to be acquired optically based on the marker element.

A further exemplary embodiment relates to a computer program product comprising instructions that, when the program is executed by a computing unit, cause said computing unit to carry out a method according to one exemplary embodiment.

A further exemplary embodiment relates to a computer-readable storage medium comprising instructions that, when the program is executed by a computing unit, cause said computing unit to carry out a method according to one exemplary embodiment.

A further exemplary embodiment relates to a method for determining an eye position and/or eye movement of an eye with an implanted ophthalmic implant that has an optically and/or electronically detectable marker element. The method comprises detecting the marker element of the ophthalmic implant in the eye, determining a position and/or movement of the marker element and ascertaining the position and/or movement of the ophthalmic implant and/or of the eye based on the determined position and/or movement of the marker element.

An ophthalmic implant here is an implant that is suitable for implantation in a patient's eye, that is to say in particular a human or animal eye. An ophthalmic implant may optionally be designed as an intraocular lens (IOL). The ophthalmic implant may have an optical part and a non-optical part, wherein the optical part may have a lens having a refractive and/or diffractive effect or may be designed as such. It may be essential here that the optical part is optically transparent and that light passing through it is preferably not deflected and/or reflected and/or absorbed by unwanted influences. As an alternative or in addition, the ophthalmic implant may have a sensor in order to ascertain one or more parameters inside the eye, such as the inner eye pressure and/or a concentration of a substance, such as sugar. The non-optical part may in particular have a haptic element or be designed as such. The fact that the non-optical part is connected to the optical part in this case means that there is mechanical contact between the optical part and non-optical part. Optionally, the optical part and the non-optical part may be formed together in one piece and form the ophthalmic implant. The fact that, as an alternative, the non-optical part is able to be connected to the optical part means here that the optical part may be assembled with the non-optical part such that they are connected to one another after assembly. Some exemplary embodiments may be designed such that an assembly of the ophthalmic implant comprises a connection of the optical part and non-optical part during the implantation process. By way of example, an ophthalmic implant may be present as a modular and/or multi-part IOL that is first assembled in the capsular bag, which comprises connecting the optical part and non-optical part.

A marker element is in this case an element that provides marking of the ophthalmic implant. Accordingly, a marker element constitutes an information carrier that is connected fixedly to the ophthalmic implant. The marker element may in this case provide optically acquirable information, such as a machine-readable character and/or a character recognizable to the human eye and/or a geometric shape, from which information is able to be extracted. Within the scope of the present disclosure, a marker element constitutes a computer-readable marker element. The marker element may in particular have a coded marker, for example in the form of a barcode, and/or an electronically readable data storage unit. The marker element may for example be applied to the ophthalmic implant and/or represent one or more characters incorporated therein and for example be installed by printing and/or engraving on and/or in the ophthalmic implant. As an alternative or in addition, the marker element may constitute a stand-alone element that is able to be inserted into the ophthalmic implant, for example by casting, and/or is able to be fastened thereto, for example by adhesive bonding. A marker element may in particular be present in the form of a coded marker or designated as such. In this case, a “coded marker” is a marker on or in the intraocular lens in which information is able to be stored/coded, that is able to be made visible using imaging methods and may thereafter also be “read” by interpreting the information obtained using the imaging methods. This is possible for example by virtue of—in the preferred variant—an appropriate physical structure of the marking or else a printed marking, that is to say a marking recognizable by color differences (including fluorescence). The fact that this information in the “coded marker” may be assigned to a precise absolute position on or in the intraocular lens also makes the “coded markers” very easy to use for alignment processes or for checking an alignment and more generally the correct position in the eye.

Information for the machine-based identification and/or characterization of the ophthalmic implant is in this case information that is able to be acquired in a machine-based manner, for example by an image acquisition device, and is able to be evaluated, for example by image evaluation. The fact that this is possible in a machine-based manner means here that the information is able to be acquired and evaluated using purely machine-based means without this necessarily requiring human intervention. In other words, the marker element is machine-readable, such that the provided information is able to be read out automatically. Likewise, the information for the machine-based determination of an alignment of the ophthalmic implant may be acquired using purely machine-based means, such that the alignment of the ophthalmic implant is able to be detected automatically based on the information.

The fact that the marker element is arranged in and/or on the ophthalmic implant means that the marker element may be partially or completely arranged on an outer surface of the ophthalmic implant and in particular attached thereto and/or partially or completely integrated into the ophthalmic implant. By way of example, the marker element may be adhesively bonded to the ophthalmic implant. As an alternative, the marker element may be cast into the ophthalmic implant. A combination of both is also possible, wherein for example one part of the marker element is cast into the ophthalmic implant and another part of the marker element is attached to an outer surface of the ophthalmic implant.

The fact that the information is able to be read out in a machine-based manner means here that the information provided by the marker element is able to be acquired by a machine-based system, without this necessarily requiring intervention from the user or surgeon. By way of example, a suitable machine-based system may have an image acquisition device, which may for instance have a camera, and a computing unit, by way of which the information is able to be optically acquired and evaluated through image evaluation.

A data storage unit here is an electrical or electronic component or an arrangement thereof that is able to provide information in electronically readable form. In particular, the electronic readout may enable contactless readout of the information without this requiring physical contact between a reader and the data storage unit. Rather, the data storage unit may be designed to provide the information using a wireless connection technology, such as by way of Wi-Fi, Bluetooth and/or by using inductive transmission technology. Information may also optionally be stored contactlessly in the data storage unit. Optionally, the data storage unit may have an RFID element, or be designed as such, which is able to be read out and/or written to contactlessly by way of a suitable RFID read and write device.

A data storage unit may optionally also be referred to as an “electronic tag” and may constitute an identifier on or in the ophthalmic implant containing information that is able be written to, re-read from and—for information for which this has been approved-modified contactlessly in this unit in electronic form. Such a data storage unit may optionally contain RFID structures. The write, read, and modification steps for information when using a data storage unit may be very easy to implement. To determine the position and/or alignment of the marker element, the data storage unit may have appropriately formed and/or precisely arranged antenna structures and/or multiple subunits of the data storage units that are precisely positioned in relation to one another, which provide access to absolute position information through appropriate positioning. In addition to these position ascertainment variants, a data storage unit in the form of an electronic chip may however also be made visually visible, and the precise absolute position thereof may thus also be used as a visual reference for positioning the ophthalmic implant.

Information in a coded marker may be created and/or written for example at the start of the process of manufacturing the ophthalmic implant and may then optionally be made visually visible and/or read out and/or interpreted over the entire lifetime of the ophthalmic implant. Information in a data storage unit may be created in the process of manufacturing the ophthalmic implant and may then optionally be read out contactlessly over the entire lifetime of the IOL, but, according to one exemplary embodiment, said information may also be modified if necessary or desired, for example for the benefit of the patient. When using a marker element in the form of a coded marker and also in the form of a data storage unit, it is possible to add and modify information in the manufacturing process, preoperatively, intraoperatively and/or postoperatively, optionally in a database (preferably in an electronic medical record (EMR system)), to which a specific ID for the respective intraocular lens, stored in the coded marker or the data storage unit, grants access.

Machine-based determination of an alignment of the ophthalmic implant here is the determination of the position and/or orientation of the ophthalmic implant relative to the patient's eye or part of the patient's eye during and/or after implantation in the patient's eye using machine-based means. This may in particular be carried out using an image acquisition device and computer-aided image evaluation. The determined alignment of the ophthalmic implant may then be provided to the surgeon in order to assist the implantation operation, for instance by a suitable output device, such as a computer display. “Machine-based” means here that the steps or the method may be performed in a partially automated and/or fully automated manner by a system and that, optionally, there is no need for intervention by the surgeon. The steps may in particular be performed by a computer-aided system. The machine-based determination of an alignment of the ophthalmic implant may in particular comprise an imaging method. An imaging method is used for instance to recognize the presence of a marker element, and the data provided by the marker element under manufacturing, intra-operative and/or postoperative imaging conditions are able to be read reliably. As an alternative or in addition, a data storage write and/or readout method is used during the manufacture of the IOL, intraoperatively and/or postoperatively, in which the data storage unit is able to be used.

The object of the disclosure is furthermore achieved by a marking unit and/or a planning and/or control unit and/or an evaluation unit that, together with corresponding imaging systems and/or data storage write and/or readout systems, marks or codes data on or in the intraocular lens, reads out data, processes data and thus controls upcoming further steps in the life of an intraocular lens, as well as by corresponding marking, planning, control and evaluation methods.

A readout apparatus here is a read and/or write apparatus by way of which information or data may be read out from and/or written to a data storage unit of an ophthalmic implant. By way of example, it may be an RFID transceiver.

A computer program product may for example be present in the form of a program code that is stored on a physical data carrier and/or is able to be downloaded via a network. A computer-readable storage medium may accordingly be present in the form of a physical data carrier or a storage unit on which a corresponding program code is stored.

Determining an eye position and/or eye movement may in this case in particular be used for eye tracking in order to track any movements of the patient's eye. This may be particularly advantageous in aftercare and/or in connection with another refractive surgical operation, in particular on the cornea, since movements of the patient's eye may in this case necessitate a possible readjustment of an ophthalmic laser system. In special cases, the cataract operation itself may of course also be assisted by an ophthalmic laser system, in particular a femtosecond laser system.

The exemplary embodiments offer the advantage of providing a marker element and optionally a coded marker for ophthalmic implants, enabling optical machine-based determination of an alignment of the ophthalmic implant and machine-based identification and/or characterization of the ophthalmic implant intraoperatively and/or postoperatively. The implantation process may thereby be assisted by the provision of information by a computer-aided system, which may optionally use an image evaluation algorithm. This in particular makes it possible to determine a positioning and/or alignment of the ophthalmic implant relative to the eye in a machine-based and in particular computer-aided manner, and to assist the surgeon with the implantation process by outputting appropriate information. This is made possible in particular by the fact that the marker element is able to be detected and read out in a machine-based manner in a state in which the ophthalmic implant is implanted in a patient's eye.

Consequently, the exemplary embodiments offer the advantage that the implantation is able to achieve better clinical results, such as better visual acuity. In particular, this may optionally achieve more precise positioning of the ophthalmic implant in the eye with respect to multiple geometric degrees of freedom, such as rotation (toric IOL), inclination, and depth or effective lens position (ELP). This is able to reduce variance in cataract surgery and allow surgeons to better standardize and optimize their surgical parameters. The risk of refractive “surprises” may also be reduced, since any user errors are able to be avoided by better guidance. By way of example, software coded in a planning or control unit may be designed to provide the surgeon with information as to whether and, if necessary, how to fine-tune the position and/or orientation of the ophthalmic implant in the eye in order to optimize the result.

The exemplary embodiments also offer the advantage of greater alignment efficiency: The surgeon optionally receives immediate feedback when the ophthalmic implant is located perfectly in the target position. This may help to reassure the surgeon and optionally eliminate the need for multiple, time-consuming checks.

The exemplary embodiments also offer the advantage of being able to achieve a more efficient workflow during the manufacture of an intraocular lens or an ophthalmic implant, and also before, during and/or after the implantation of the ophthalmic implant. The marker element may in this case not only contain relevant information or data for the automatic identification, but may optionally also enable precise tracking of the position of the ophthalmic implant during the operation and during the healing process. Optionally, if necessary, an ophthalmic implant according to one exemplary embodiment may also facilitate realignment of the ophthalmic implant in the event of postoperative movements of the ophthalmic implant. The high degree of identification certainty also assists processes associated with the cataract operation, such as reordering the ophthalmic implant.

In addition, the exemplary embodiments offer the advantage that the machine-based optical detection of the marker element and the determination of the information provided by the marker element are possible, according to some exemplary embodiments, using conventional image acquisition devices. By way of example, the image acquisition device may have a conventional video camera system for optical detection of the marker element. This thus offers the advantage that there is no need for expensive additional hardware to determine the information provided by the marker.

Traditionally, no OCT is required in cataract surgery. In the exemplary embodiments disclosed here as well, no OCT system is required to determine a position and/or alignment of the ophthalmic implant relative to the patient's eye, although a combination with an OCT is possible according to exemplary embodiments.

The exemplary embodiments thus offer the advantage of significantly improving the quality able to be achieved in cataract surgery and aftercare by using marker elements in ophthalmic implants according to the exemplary embodiments and corresponding systems.

Optionally, the marker element is designed such that the information provided by the marker element is visible or detectable both to the human eye and in a machine-based manner. This offers the advantage that said information may be used by a system to provide machine-based assistance with an implantation, and may also be used directly to guide a surgeon. As an alternative or in addition, the marker element is designed so as to reliably enable machine-based acquisition of the information even under conditions in which only part of the ophthalmic implant is visible, for example in the case of a small pupil of the patient's eye, in which the iris covers part of the ophthalmic implant. As an alternative or in addition, the marker element of an IOL may be optimized for an illumination coaxial with the optical axis of the IOL, in order, for instance when the coaxial illumination is incident, to have a particularly high reflectivity and/or to be able to be detected particularly well in a machine-based manner.

The marker element may in this case be formed in one part or multiple parts. In this case, one and the same part may provide both the information for the machine-based identification and/or characterization of the ophthalmic implant and the information for the machine-based determination of an alignment of the ophthalmic implant. According to another exemplary embodiment, different parts of the marker element may provide the information for the machine-based identification and/or characterization of the ophthalmic implant or the information for the machine-based determination of an alignment of the ophthalmic implant.

Optionally, the cataract operation, at the end of which an IOL is implanted, may comprise capsulorhexis performed by way of an ophthalmic laser system in order to open the capsular bag of the lens of the eye. If necessary, further steps of a cataract operation may also be carried out by way of the ophthalmic laser system, preferably by way of a femtosecond laser system. In this case, for instance, two or more markings may be placed in the eye by way of the laser, with which a toric IOL may then be aligned, for instance. Such markings may optionally also be used to determine a relative alignment of the ophthalmic implant and the patient's eye by way of the marker element.

Optionally, the marker element is arranged in and/or on the ophthalmic implant such that it is only optically detectable when a pupil of the patient's eye is dilated. Typically, the diameter of an adult's pupil is between 2 mm and 4 mm in bright lighting conditions and between 4 mm and 8 mm in dark lighting conditions. However, the average pupil diameter may also be influenced by the patient's age and refractive status in addition to the lighting conditions. A range within which the pupil diameter lies therefore typically extends from around 2.5 mm to around 6 mm. In particular, it may be advantageous to arrange the marker element outside a central diameter of 4.4 mm, since this range is prescribed by relevant ISO standards for conventional toric markings as well. Typically, IOLs have an overall diameter of the optical part of 5 mm to 6.5 mm. The marker element may optionally be arranged in the edge region of the IOL, for instance outside a diameter of 4.4 mm of the central optical part, or the marker element may be extended to the entire optical part or the entire IOL.

This offers the advantage that the marker element does not affect the optical properties of the ophthalmic implant in the event of a non-artificially dilated pupil, and thus does not affect the patient's visual perception. Nevertheless, the marker element is accessible or optically detectable if necessary when the pupil of the eye is dilated.

Optionally, the marker element is arranged in and/or on the ophthalmic implant such that the information for the identification and/or characterization of the ophthalmic implant is also able to read out in a machine-based manner and the information for the machine-based determination of the alignment of the ophthalmic implant is also optically acquirable preoperatively and/or intraoperatively and/or during manufacture of the ophthalmic implant. This offers the advantage that the information is provided over the entire lifetime of the ophthalmic implant and/or of the patient and is able to be read out if necessary. This also offers the advantage that the information carrier is permanently linked to the ophthalmic implant and accordingly cannot be lost nor incorrectly assigned. This optionally offers the advantage of enabling identification and/or characterization of the ophthalmic implant over the entire lifetime of the ophthalmic implant. The information provided by the marker element may in this case optionally be used during manufacture, checking, packaging, delivery, preparation for implantation (preoperatively), during implantation (intraoperatively) and after the operation, for instance in follow-up examinations (postoperatively). This makes it possible, at any time, to identify and/or characterize and optionally determine the alignment of the ophthalmic implant relative to the patient's eye. The exemplary embodiments also offer the advantage that it is possible, postoperatively, in a machine-based manner and contactlessly, to check which ophthalmic implant has been implanted. This may be advantageous for example if it is necessary to provide corresponding evidence of the identification and/or characterization of the ophthalmic implant in legal proceedings, in the event of complaints and/or for quality management. Accordingly, the ophthalmic implant may optionally be designed such that the information for the identification and/or characterization of the ophthalmic implant and the information for the machine-based determination of the alignment of the ophthalmic implant is able to be provided not only intraoperatively and/or postoperatively, but also preoperatively and/or during a manufacturing and/or packaging process. The information may in this case be read out electronically and/or optically.

Optionally, the marker element has a marking in the form of a machine-readable code, wherein the information for the machine-based identification and/or characterization of the ophthalmic implant is stored in the machine-readable code. This offers the advantage of being able to read out the information from the marker element through simple image evaluation. By way of example, the machine-readable code may have a serial number and/or other unique identification numbers based on which the ophthalmic implant is able to be uniquely identified and/or characterized. As an alternative or in addition, the machine-readable code may provide one or more network addresses at which information about the ophthalmic implant may be provided. This offers the advantage that the information or amount of data to be provided in the marker element may be selected to be very low, while a significantly larger amount of data and a large amount of information may be provided at the network address. This also offers the advantage that the data stored on a data memory located at the network address may be expanded and/or updated in order to add for instance additional information about the implantation process and/or postoperative examinations. As an alternative or in addition, a size and/or position and/or alignment of the marking may provide the information for the machine-based determination of the alignment of the ophthalmic implant. This offers the advantage that a marker element may be used to fulfill multiple functions, and the outlay and the costs for the provision of the marker element or marker elements may accordingly be kept low.

Optionally, the machine-readable code has a one-dimensional barcode and/or a two-dimensional matrix code and/or a three-dimensional matrix code or consists thereof. This offers the advantage that the machine-readable code is able to be provided in a particularly simple way. By way of example, such a machine-readable code may be engraved into the ophthalmic implant or printed thereon.

Optionally, the machine-readable code may have a dot matrix. The dot matrix may in this case have marking dots, which may have a pseudo-random irregular character. Optionally, the dot matrix may be arranged in the optical part or in the optically effective zone of the ophthalmic implant. The coded dot matrix may be constructed in this case from the marking dots such that a virtual polar or Cartesian base grid is arranged on the optical part, such that this base grid may be used to describe similar sectors or similar cells each having a defined base grid point of the sector or the cell. In this case, a real marking dot of the dot matrix is arranged in each sector or each cell at a position that has a respective offset to the associated camera base grid point, the offset in each sector or each cell running in one of four possible directions, which preferably run pairwise opposite to one another and having a defined distance to the base grid point. This provides four states. A fifth state may be defined for instance by the absence of a marking dot at one of the four options around a base grid point. Further states may also be provided by other distances and/or offset directions.

Optionally, the marker element comprises a data storage unit, or consists thereof, which is designed to provide the information for the machine-based identification and/or characterization of the ophthalmic implant in the form of electronically readable data. This offers the advantage that a large amount of data is able to be provided by a marker element with very small spatial dimensions. In particular, this offers the advantage that the spatial dimensions of the marker element are able to be decoupled from the amount of data to be stored thereon. By way of example, the marker element may have an RFID element or consist thereof. This offers the advantage of being able to use conventional RFID read and write devices to write to and/or read out the marker element. In addition, this offers the advantage of not having to provide an internal power supply in the marker element or in the patient's eye.

Optionally, the data storage unit is designed and arranged in and/or on the ophthalmic implant such that the alignment of the ophthalmic implant is able to be determined based on a position and/or alignment of the data storage unit. This offers the advantage that the optically acquirable properties of the data storage unit provide fixedly stored information with regard to the alignment of the ophthalmic implant, and this information may then be used to determine the alignment and/or positioning of the ophthalmic implant relative to the patient's eye or part thereof. This thus offers the advantage that it is not necessarily necessary to provide a further part of the marker element, such as a marker, in order to provide the information with regard to the position or alignment of the ophthalmic implant.

Optionally, the method for the machine-based determination of an alignment of an ophthalmic implant is designed such that the determination of the alignment of the ophthalmic implant relative to the patient's eye is repeated multiple times and optionally carried out continuously. This offers the advantage of enabling continuous computer-aided monitoring of the alignment and/or position of the ophthalmic implant relative to the patient's eye, and the surgeon thereby receives real-time assistance when implanting the ophthalmic implant.

Optionally, the method furthermore comprises reading out information for the machine-based identification and/or characterization of the ophthalmic implant from the marker element. This offers the advantage that the identification and/or characterization may also take place in an automated manner, and the risk of implantation of an incorrect and/or unsuitable implant in the patient's eye may accordingly be reduced. By way of example, the method may comprise outputting a warning notification to the surgeon if the identification and/or characterization comes to the conclusion that the implant provided for the implantation is not suitable and/or does not meet any stored specifications. Optionally, the marker element in this case has a marking in the form of a machine-readable code or consists thereof. In this case, the readout of the information for the machine-based identification and/or characterization of the ophthalmic implant from the marker element may comprise reading out the information from the machine-readable code. The machine-readable code may in this case have a one-dimensional barcode and/or a two-dimensional matrix code and/or a three-dimensional matrix code or consist thereof.

Optionally, the marker element comprises a data storage unit or consists thereof. The readout of information for the machine-based identification and/or characterization of the ophthalmic implant may in this case comprise electronic readout of data from the data storage unit containing the information for the machine-based identification and/or characterization of the ophthalmic implant. The machine-based determination of the position and/or alignment of the marker element relative to the ophthalmic implant may comprise determining a position and/or orientation of the data storage unit.

Optionally, a system for providing machine-based assistance with the implantation of an ophthalmic implant in a patient's eye may be designed such that the image acquisition device comprises a camera and optionally a microscope. This offers the possibility of reliably optically detecting even small structures of the marker element and/or small marker elements. Optionally, the microscope may be designed as a surgical microscope. This offers the advantage that a surgical microscope is often used for the implantation of the implant in any case, and accordingly no further microscope has to be provided for providing machine-based assistance with the implantation. The requirements in terms of additional hardware to be provided for the system may accordingly be kept low.

Optionally, the output unit of the system has a display device, in particular a display. This offers the advantage that the information about the determined alignment of the ophthalmic implant relative to the patient's eye may be displayed to the surgeon in a simple and easily visible way.

The method for manufacturing an IOL may be designed in this case such that arranging the marker element comprises affixing a machine-readable code, wherein the machine-readable code optionally has a one-dimensional barcode and/or a two-dimensional matrix code and/or a three-dimensional matrix code or consists thereof. This offers the advantage that the marker element is able to be provided in and/or on the intraocular lens particularly inexpensively. This may also offer the advantage that the marker element is able to be affixed only after the other manufacturing steps are complete. It is thereby possible for instance to dispense with affixing the marker element if one or more previous manufacturing steps have led to a faulty IOL. It is also possible for instance to take into account information in the marker element that may not have been available in the previous manufacturing steps, for example information concerning the patient.

As an alternative or in addition, the arrangement of the marker element comprises or consists in installing a data storage unit that contains the information for the machine-based identification and/or characterization of the ophthalmic implant in the form of electronically readable data. This offers the advantage that it is optionally possible to store a larger amount of data on the data storage unit than on a machine-readable code. This also optionally offers the possibility of being able to expand and/or modify the data stored on the data storage unit if desired. To this end, the data may be accessed for example by way of a write-read device. Optionally, the data storage unit may have an RFID element and the write-read device may be designed as an RFID write-read device.

The use of a marker element, that is to say a marking in the form of a machine-readable code and/or a data storage unit, may in this case optionally comprise one or more of the following applications:

The use of the provided machine-based identification and/or characterization information for identification and/or characterization during manufacture and/or packaging of the ophthalmic implant. This may be used for example, once the marker element has been installed in and/or on the ophthalmic element, to enable seamless traceability and/or constant identifiability of the ophthalmic implant. In particular, this may be advantageous since it is possible to check the correctness of an assignment of ophthalmic implant and associated packaging and/or of associated documents.

The use of the provided machine-based identification and/or characterization information preoperatively for assignment and/or checking of an assignment of the ophthalmic implant to a patient's eye. This may be used for instance to ensure the correct assignment of the ophthalmic implant to the patient's eye immediately before the implantation and to reduce the risk of implantation of an incorrect ophthalmic implant.

The use of the provided information for the machine-based determination of a position and/or alignment of the ophthalmic implant intraoperatively for machine-based determination of the alignment of the ophthalmic implant relative to the patient's eye in order to assist the implantation process. This makes it possible to facilitate the alignment of the ophthalmic implant relative to the patient's eye and to reduce the risk of an incorrect implantation.

The use of the provided information for the machine-based determination of an alignment of the ophthalmic implant postoperatively for machine-based determination and/or checking of the alignment of the ophthalmic implant relative to the patient's eye. This offers the advantage that, even as part of aftercare and/or in the event of the patient being subjectively dissatisfied with the implantation result, it is possible to carry out a check of the alignment of the ophthalmic implant relative to the patient's eye and accordingly to compare the target alignment with an actual alignment in a machine-based manner.

The use of the provided postoperative machine-based identification and/or characterization information for identification and/or characterization of the ophthalmic implant and/or for checking of an assignment of the ophthalmic implant to the patient's eye in which the ophthalmic implant is implanted. This offers the possibility of identifying and/or characterizing the implanted ophthalmic implant beyond doubt even after implantation and of accordingly ascertaining a match or discrepancy between planned and actual implantation. This may be particularly advantageous for checking a correct implantation if the correct implantation is called into question.

The features and exemplary embodiments specified above and explained below should not only be considered to be disclosed in the respective explicitly mentioned combinations in this case, but are also comprised by the disclosure in other technically advantageous combinations and exemplary embodiments. In particular, features that are described with reference to one exemplary embodiment may also be implemented in other exemplary embodiments.

Further details and advantages are now intended to be explained in more detail based on the following examples and exemplary embodiments with reference to the drawings, without the exemplary embodiments however being limited thereto.

### BRIEF DESCRIPTION OF THE DRAWINGS

### In the Drawings:

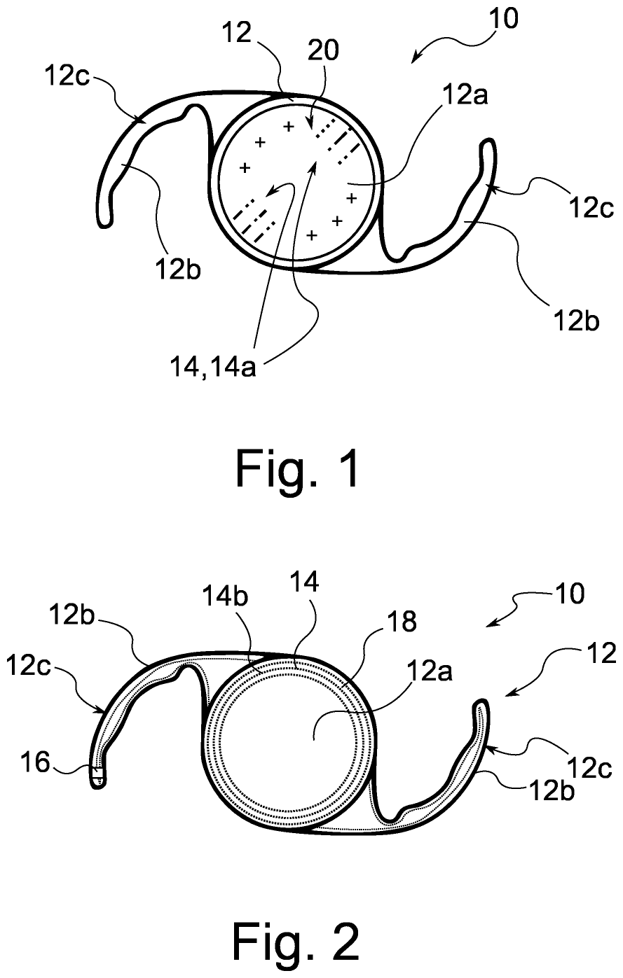

FIG. 1 shows a schematic illustration of an intraocular lens having a marker element according to one exemplary embodiment;

FIG. 2 shows a schematic illustration of an intraocular lens having a marker element according to a further exemplary embodiment;

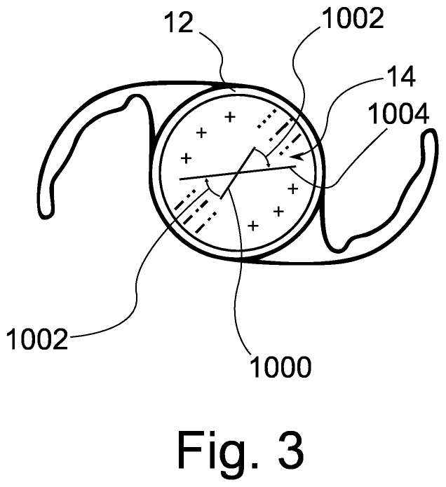

FIG. 3 shows an exemplary visualization of an IOL for assisting an implantation process;

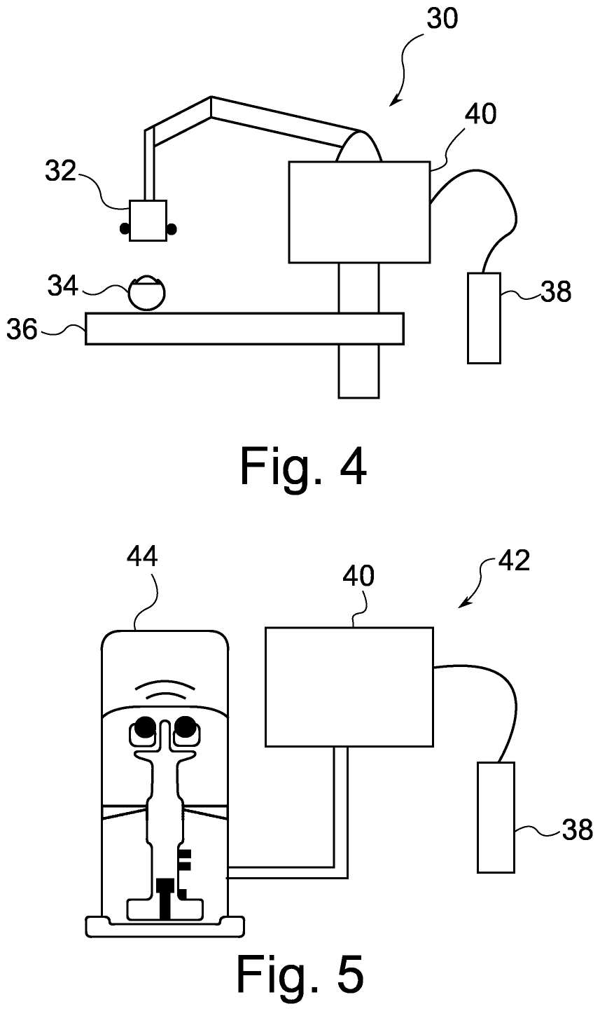

FIG. 4 shows a system for providing machine-based assistance for a user when implanting an ophthalmic implant according to one exemplary embodiment;

FIG. 5 shows a system for exchanging information with an ophthalmic implant according to one exemplary embodiment;

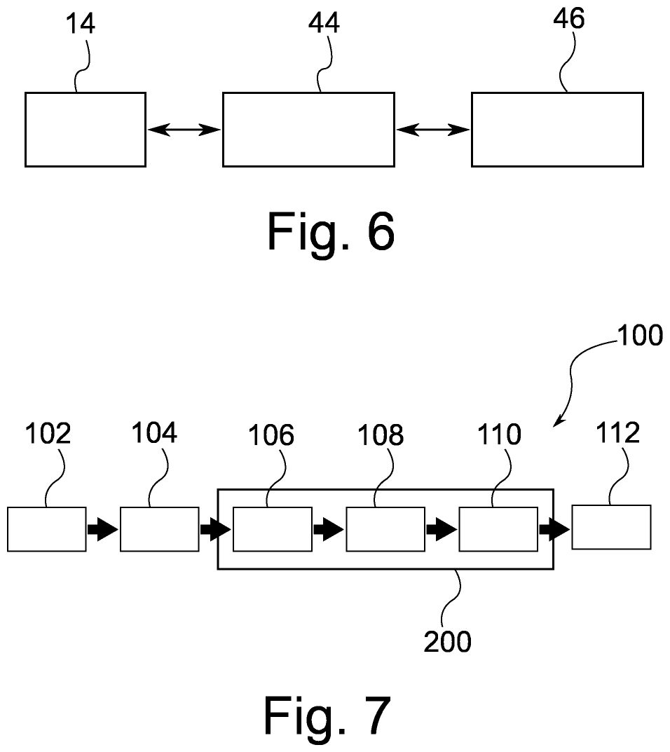

FIG. 6 shows a schematic illustration of the interaction of a marker element of an ophthalmic implant with a write and/or readout apparatus and a separate information source; and

FIG. 7 schematically shows, by way of example, a flowchart of various optional use cases of a marker element of an ophthalmic implant over the lifetime of the ophthalmic implant.

### DETAILED DESCRIPTION OF EXEMPLARY EMBODIMENTS

According to one exemplary embodiment, provision is made for an ophthalmic implant 10 in the form of an intraocular lens 12 (IOL).

FIG. 1 shows an exemplary embodiment of an ophthalmic implant 10 that is formed from an IOL 12. The IOL 12 in this case has an optical part 12a formed by a lens, and a non-optical part 12b having two haptic elements 12c. The IOL 12 also has a marker element 14 that provides information, able to be acquired in a machine-based manner, for the identification and/or characterization of the IOL 12, and additionally provides information, able to be acquired in a machine-based manner, about the positioning and/or alignment of the IOL 12. In other words, the marker element 14 is machine-readable, such that the information provided by the marker element 14 is able to be extracted and evaluated in a machine-based manner.

According to the exemplary embodiment illustrated in FIG. 1, the marker element 14 comprises a coded marker 14a that comprises multiple markings. In this case, the marker element 14a comprises a line and dot pattern running parallel to the diameter of the IOL 12, which pattern, on the one hand, through its arrangement, provides information regarding the position and/or alignment of the IOL. To this end, for example, the line and dot pattern may be arranged such that an axis position of any toric axis of the IOL 12 is able to be derived therefrom. On the other hand, the arrangement of the lines and dots and/or their length, size and/or color may be used to code information that is able to be evaluated in a machine-based manner and accordingly constitutes a machine-readable code 20. By way of example, the information may be stored in the line and dot pattern in the manner of a barcode and provide machine-readable information for the identification and/or characterization of the IOL 12. In addition, the marker element of the IOL 12 may also have further, additional markings, which are arranged as cross-shaped markings along the outer edge of the optical part 12a in FIG. 1 by way of example. These markings may be used for instance to determine a center point and/or other geometric points and/or properties of the IOL 12 in a machine-based manner. By way of example, the markings of the marker element 14 may be applied to the IOL 12. This may be achieved for example by printing and/or engraving.

The marker element 14 and all associated markings are arranged in this case on and/or in the IOL 12 such that they do not interfere with the optical properties of the IOL 12 during typical use thereof. In particular, the marker element may be arranged at the edge of the optical part such that it is covered by the iris in a normal state of the patient's eye in an implanted state, and accordingly does not affect the light entering the eye through the pupil. In order also to make the marker element 14 accessible for optical detection in an implanted state, the marker element 14 may be arranged for instance such that it is at least partially optically detectable in the case of a medicinally dilated pupil of the patient's eye and allows the provided information to be extracted.

FIG. 2 shows an ophthalmic implant 10 in the form of an IOL 12 according to one exemplary embodiment, in which the marker element 14 has a data storage unit 14b. The data storage unit 14b in this case comprises a chip 16 on which in particular information in the form of electronic data is able to be stored electronically. The data storage unit 14b furthermore has an antenna structure 18 that is connected to the chip 16. The data storage unit 14b is able in this case to be read out and/or written to in a machine-based manner and optionally contactlessly using a suitable read and write device. By way of example, the data storage unit may be designed as an RFID element and accordingly be read out and written to contactlessly. The data stored in the chip 16 may in particular comprise information for the identification and/or characterization of the IOL 12. Furthermore, the antenna structure 18 may be arranged in and/or on the IOL such that it is optically detectable in a machine-based manner in the case of a dilated pupil and may be used to position and/or orient the IOL 12 relative to the patient's eye. By way of example, the antenna structure may be designed and arranged such that the center point of the optical part 12a of the IOL 12 is able to be derived therefrom.

In addition, according to one exemplary embodiment, provision is made for a system for providing machine-based assistance with the implantation of an ophthalmic implant, for example the IOL 12, which assists the surgeon with IOL positioning. In particular, the method offers the surgeon immediate intraoperative feedback in order to optimize IOL positioning. In this case, the IOL has a marker element 14 that, according to the exemplary embodiment shown, is formed as a coded marker on the intraocular lens 12. The marker element 14 allows not only reliable identification of the IOL through information provided by the marker element, but also makes it possible to determine the position and/or alignment of the marker element, based on which it is possible to derive the position and/or alignment of the IOL relative to the patient's eye during the operation. The information provided by the marker element enables new possibilities for computer-aided optimization of IOL positioning. In addition or as an alternative, the marker element may have one or more data storage units 14b each having at least one corresponding antenna structure 18 that are able to provide information about the position and/or alignment of the IOL. In addition to these position ascertainment variants, a data storage unit in the form of an electronic chip may however also be made visually visible, and the precise absolute position thereof may thus also be used as a visual reference for positioning and/or aligning the IOL.

The systems and methods in the described exemplary embodiments are described here for a corresponding intraocular lens 12 that is provided with a marker element that has a coded marker and/or a data storage unit. According to further exemplary embodiments, however, the disclosure content also covers other ophthalmic implants that are able to be positioned in the eye, such as a capsule clamping ring, stents and ICLs (implantable contact lenses), which are provided with a marker element according to the same or a similar principle and may be positioned accordingly.

The coded marker on an intraocular lens 12 is formed, according to one exemplary embodiment, by a machine-readable pattern or a machine-readable code 20. Such a pattern is optionally recognizable under various types of illumination, such as for example, but without limitation, standard white light illumination, fluorescent illumination, laser illumination, etc. The coded marker is positioned on an IOL 12 such that it is able to be detected during the surgical implantation and optionally also after the operation. Optionally, the coded marker is positioned at the periphery of an optical part 12a of the IOL that is usually accessible by way of a pupil dilation (see one exemplary embodiment in FIG. 1).

The marker element 14 contains information, such as specification data in relation to the individual ophthalmic implant 10 (in the case of an IOL, for example, diopter, type, manufacturer, model, material, toric axis). However, the specification data may also be represented by a unique identifier that enables the data to be retrieved from a database.

The marker element 14 furthermore constitutes information about geometric data of the individual implant enabling computer-aided recognition of its position and/or alignment. Due to the coded nature of the marker element, even a subset of recognized features of a coded marker may provide useful information to enable reliable positioning and/or alignment of an IOL 12 in the patient's eye. The marker element 14 optionally contains error recognition means, error tolerance means and, ideally, error correction means.

Generally speaking, a marker element on an IOL 12 may contain any kind of visually recognizable coded information that offers the functionality described above. Examples of types of coding include (i) standard codes such as linear barcodes or matrix (2D) barcodes including dot code, QR code, or (ii) advanced codes such as 3D matrix codes. Machine-readable codes may vary in terms of the number, size or width of (individual) elements (for example pixels or lines of a barcode), the total code size, the distance between the elements and/or the alignment of the elements within the code. With regard to specific exemplary embodiments of marker elements or data storage units on an intraocular lens, reference is made to further documents from the applicant, the contents of which are to be incorporated here by reference: DE 10 2020 214 126 and DE 10 2021 121 166.

Such marker elements may for example offer the following advantages:
(i) Assistance functions for intraoperative positioning of IOLs during a cataract operation based on marker elements the positions of which are detectable. If the marker element comprises a data storage unit, for example, the optical acquisition of the information for the positioning and/or alignment may comprise visual detection of an electronic chip and/or of an antenna structure or part thereof.
(ii) Analysis and visualization functions in relation to an IOL position after a cataract operation based on a coded IOL marker or, if applicable, data storage units with antenna structures or multiple data storage subunits of a marker element, the positions of which are detectable, or through visual detection of an electronic chip belonging to the data storage unit.

The use of a marker element enables the provision of IOL design specification data (including IOL geometry) and actual IOL position data. Based on the data provided by the marker element, the following functionalities may for instance be enabled:
a) reading out, visualizing and/or documenting IOL data;
b) checking, identifying and/or confirming that the actual IOL matches the IOL intended for implantation;
c) assisting the toric alignment of an IOL, that is to say the alignment of the cylinder axis of a toric IOL;
d) optionally: assisting the:
   i) centering of an IOL;
   ii) tilting or inclination positioning of an IOL;
   iii) depth positioning of an IOL (along the optical axis of the patient's eye)
   iv) assistance for setting an imaging focus and/or zoom control of an image acquisition system.

By virtue of using a marker element on the IOL optics, that is to say on an optical part of the IOL, the abovementioned functionalities are available during intraoperative and postoperative workflows. Conventional computer-aided surgery systems do not offer any assistance and, in particular, any comprehensive guidance on how to precisely align the IOL to the planned target position in the patient's eye (usually only rotation and centering targets are displayed for the IOL), or are even a “black box” the exact function of which is beyond the surgeon's knowledge, and therefore offer only very limited repeatability for the surgeon. In particular, traditionally, only implicit information about the IOL position relative to the patient's eye is provided through refractive wavefront measurements, on the basis of which only limited precision is achievable, since, due to varying surgical parameters and the unnatural state of the patient's eye due to the fixation during the operation, it is typically not possible to set the exact same parameters for different implantation processes. Conventional solutions therefore support the pre-implantation stage and an early phase of the implantation. However, conventional procedures cannot be reliably performed in a computer-aided manner, in particular when the IOL is implanted in a patient's eye: This deficiency in conventional procedures is eliminated by the use of a machine-detectable marker element having a coded marker and/or a data storage unit according to the present disclosure.

Ophthalmic implants and systems for providing machine-based assistance with the implantation of such an ophthalmic implant may, in various exemplary embodiments, have different features and offer different functions.

Some exemplary embodiments have at least the following features:
a) an ophthalmic implant having at least one marker element that provides information for the machine-based identification and/or characterization of the ophthalmic implant and information for the machine-based determination of an alignment of the ophthalmic implant. To this end, the marker element may have a coded marker and/or a data storage unit that are visible and detectable, that is to say optically acquirable, intraoperatively and postoperatively and enable at least error recognition;
b) an imaging method for recognizing the presence of the marker element and reliably acquiring the data provided by the marker element under manufacturing, preoperative, intraoperative and/or postoperative imaging conditions, and optionally corresponding methods for describing, reading out, evaluating and/or modifying the data provided by the marker element (for example from a data storage unit);
c) application software that is designed for example to evaluate the image data, acquired using the imaging method, from the marker element and to evaluate the information provided by the marker element;
d) a computer system for importing data, exporting data, computing and/or processing data, which may be compared with the evaluated data;
e) means for communicating results and giving feedback to the user, for example to the surgeon, for example in visual, auditory and/or haptic form. By way of example, these means may comprise an output unit, which may for example comprise a display.

According to one exemplary embodiment, the system may additionally enable checking of an intraoperative IOL positioning and/or alignment and postoperative IOL position analysis. To this end, the system may also have the following elements:
a) such a configuration and/or positioning of the marker element, based on which the actual positioning and/or alignment of the IOL are/is able to be determined (for example center, physical edge, toric axis, unique visual identification pattern);
b) information provided by the marker element, representing a specification of the IOL design and/or enabling access thereto (that is to say for example geometric dimensions, center, edge);
c) reference data specifying biometrics, geometry and/or properties of the patient's eye;
d) optionally: operation planning data for IOL positioning and/or alignment (for example target refraction, toric axis, etc.).

In order to enable intraoperative characterization and/or identification of the IOL, that is to say for instance to check the correct presence of the intended IOL before and/or during implantation, it may also be advantageous for the system to have the following features:
a) provision of information by or via the marker element, for example in the form of coded data that represent an IOL identification of the implanted intraocular lens and/or enable access to associated data stored elsewhere;
b) provision of information by or via the marker element containing operation planning data that contain the IOL identification of the planned intraocular lens (for example cataract planner or connection to an EMR system, that is to say an electronic medical record).

Furthermore, the data provided by the marker element may optionally contain the following information, and the properties of the marker element may be formed as follows:
a) information regarding the specification of the IOL design in the coded data; in particular a key or information regarding the unique IOL identification;
b) ability to recognize the marker element by way of OCT imaging, such that the OCT imaging may be used to carry out a visual check and/or a plausibility check of the IOL positioning and/or alignment. As an alternative or in addition, a wavefront measurement may be performed to check and/or check the plausibility of the positioning and/or alignment of the IOL. This may also be used to optimize the refractive result.

In this case, the data content of the marker element, that is to say for instance of the coded marker and/or of the data storage unit, may vary depending on the use case or depending on the exemplary embodiment. In this case, different coding techniques and/or designs of the coded marker and/or different exemplary embodiments of the data storage unit and in particular of antenna structures of the data storage unit may also be possible. Readout techniques for determining the information provided by the marker element may also vary between different exemplary embodiments. These may comprise for instance an imaging method for detecting the presence of a coded marker and for the reliable readout of the data from the coded marker and/or a method for the contactless electronic recognition and for the readout of the data storage unit.

The exemplary embodiments thus offer the advantage that more machine-readable information about the IOL is able to be provided and/or used in the intraocular lens manufacturing process, the preoperative, intraoperative and/or postoperative workflow. It is thereby possible to provide new, additional lens positioning functionalities in addition to the existing toric lens implantation, and existing steps of a conventional implantation procedure (for example toric alignment, lens confirmation) may be made easier and/or more reliable and robust.

Some of the functions enabled by this disclosure for the positioning and/or alignment (centering, inclination, depth) of an ophthalmic implant and/or for checking same may be advantageous when implanting both toric and non-toric IOLs.

An explanation is given below of a system and a method for providing machine-based assistance with the implantation of an ophthalmic implant in a patient's eye according to one exemplary embodiment:

The system and the method may be designed in particular for computer-aided IOL data processing and IOL positioning during cataract surgery based on at least one marker element.

To this end, provision is made for a system for recognizing and identifying marker elements, in particular coded IOL markers. Methods for performing data processing, visualization, documentation, IOL identification and IOL positioning based on the coded IOL marker are also provided.

The system is used here to assist the implantation of an ophthalmic implant (for example IOL) having a marker element able to be detected in a machine-based manner, having a coded marking (a coded marker). The system in this case comprises
a) an ophthalmic visualization system (for example a surgical microscope)
b) an image acquisition device, such as a camera system;
c) application software for evaluating information regarding
   (i) IOL data processing, visualization, documentation,
   (ii) IOL positioning,
   (iii) IOL confirmation or identification, and
   (iv) management of operation data, including planning data;
d) a computer system for importing data, exporting data, computing and processing data;
e) means for communicating results and for presenting feedback to the user or surgeon, for example in visual, auditory and/or haptic form
f) optionally: an IOL database containing implant specifications;
g) optionally: an imaging/measurement technique, such as OCT or wavefront aberrometry;
h) optionally: a connected electronic medical record (EMR) containing information about the patient, their eye condition and the operation plan.

The system is designed such that it enables the following functionalities:
a) reading out, visualizing and documenting IOL data from the marker element;
b) optionally: feedback from the comparison of the actual IOL with the intended IOL;
c) optionally: an assistant for assisting the:
   (1) toric alignment of an IOL
   (2) centering of an IOL
   (3) tilt positioning of an IOL
   (4) depth positioning of an IOL
   (5) setting of the imaging focus and/or control of the zoom
d) optionally: automated business processes enabled by the reliable IOL readout, such as for example automatic IOL reordering.

The assistants for assisting the positioning and/or alignment of the IOL, that is to say for toric, centering, tilting and/or depth positioning of an IOL, are designed here such that the surgeon is able to efficiently assess/evaluate the result of the IOL implantation. Visual and/or acoustic feedback is possible during alignment in order to indicate whether the result is within the planned or best practice parameter range.

If the marker element or the marker elements comprises or comprise a data storage unit as an alternative or in addition to coded markers, then the system requires a write and/or read device for reading out the information from the data storage unit. If the data storage unit is used instead of the coded markers and if positioning functions are to be used, then it is necessary to use specially designed antenna structures and/or a specially designed data storage unit and/or multiple subunits, positioned precisely relative to one another, of the data storage unit to determine the positioning and/or alignment of the ophthalmic implant.

According to other exemplary embodiments, a data storage unit in the form of an electronic chip may be made visually visible, and the precise absolute position thereof may thus also be used as a visual reference for positioning and/or aligning the IOL.

The functionalities of such an exemplary embodiment are described in detail below.
a) Reading out, visualizing and documenting the IOL data:

The system is able to recognize the presence of the marker or data storage unit based on the coded marker or the data storage unit of the marker element, and
a) reading out the IOL data (including the position information, where applicable) provided by the marker element or importing, where applicable, the implant specification from the IOL database;
b) applying error recognition/correction to the coded data;
c) visualizing the IOL data for the user in textual or graphical form,
d) documenting the IOL data in electronic form.
b) Confirming the actual IOL and comparing it with the planned IOL:

The system uses an imaging method or an electronic readout method to import or extract the IOL design specification derived from the coded marker or the data storage unit. Based on the IOL design specification from the coded marker or the data storage unit and/or the IOL database, and the intraocular lens from the operation plan, as provided in accordance with the operation plan, the system generates an electronic output of information for the surgeon or user indicating that either the correct IOL has been selected for implantation (IOL confirmation) or that an incorrect IOL has been selected for implantation (IOL mismatch). In the event of an IOL confirmation, the system outputs a message to the user that the IOL has been confirmed. In the event of an IOL mismatch, the system outputs a warning to the user and asks for the selected and planned IOL to be checked.
c) Assistant for assisting the toric alignment of an IOL:

The system uses an imaging method to extract the toric axis of the IOL from information provided by the marker element, in particular from information provided by the coded marker. As an alternative or in addition, the system uses a reader to read out the corresponding data contactlessly from the data storage unit of the marker element, which data storage unit is designed in this case so as to enable position detection. An electronic chip contained in the data storage unit may alternatively also be detected visually with regard to its precise absolute position. Based on
a) the reference data specifying the biometrics, geometry and properties of the eye (optionally including femtosecond laser capsule markings), and
b) the toric axis of the IOL, the system usually generates an electronic output of information to the user in order to control the correct alignment of a toric IOL. The system gives the user direct feedback, for example in visual form (as illustrated by way of example in FIG. 3) and/or in acoustic form.

FIG. 3 illustrates, by way of example, a visualization of an IOL 12 as may be displayed for instance to a surgeon to assist the implantation process. In this case, based on the information provided by the marker element 14, the alignment and position of the toric axis of the IOL 12 is determined in a machine-based manner and displayed as an axis 1000 together with a visualization of the IOL 12. Furthermore, visualized arrows 1002 indicate a direction of rotation along which the IOL 12 is to be rotated in order to bring the toric axis 1000 of the IOL 12 into line with the axis position of the astigmatism axis 1004 of the patient's eye. The astigmatism axis 1004 is likewise visualized in order to provide visual assistance.

One special feature here is that the data of the coded marker or of the data storage unit provide the physical position of the toric axis of the IOL within the operation scene and at least offer error recognition that ensures that the system correctly recognizes the information provided by the marker element with regard to the toric axis.

FIG. 4 shows a system 30 for providing machine-based assistance for a user when implanting an ophthalmic implant according to one exemplary embodiment. In this case, the system 30 has a surgical microscope 32 that is used when implanting an ophthalmic implant 10 in a patient's eye 34. The patient's eye 34 is illustrated symbolically here as lying on a patient table 36, although, during actual use, the patient's eye 34 obviously forms part of the patient (not shown). The surgical microscope 32 serves here as an image acquisition device for acquiring images of at least part of the patient's eye 34 and of at least one marker element 14 of the ophthalmic implant 10 during the implantation process in which the ophthalmic implant 10 is implanted in the patient's eye 34.

In addition, the system 30 has a computing unit 38 that is connected to the image acquisition device, that is to say to the surgical microscope 32, and is configured to determine a relative alignment of the ophthalmic implant 10 relative to the patient's eye 34 based on the acquired images of the patient's eye 34 and of the marker element 14. By way of example, the computing unit 38 may be designed as a control unit and/or as a computer or comprise same.

The system 30 furthermore has an output unit 40 that is configured to output information about the determined alignment of the ophthalmic implant 10 relative to the patient's eye 34 to a user. To this end, the output unit 40 may have corresponding output elements for outputting a visual and/or acoustic and/or haptic signal to the user. By way of example, the output device 40 may have one or more screens or displays for an image output and/or have one or more loudspeakers for an audio output. By way of example, the system 30 may be configured such that, in order to assist the user in the implantation process via the output unit 40, a relative alignment of the ophthalmic implant 10 relative to the patient's eye 34 and/or instructions for reducing any deviation of the target alignment from the actual alignment are output.

The system 30 may furthermore be configured to read out information for the identification and/or characterization of the ophthalmic implant 10 from the marker element 14. To this end, an identification number may for instance be read out from the marker element and taken as a basis for uniquely identifying the ophthalmic implant 10 preoperatively and/or intraoperatively. Optionally, the system 30 may be configured to compare the identification and/or characterization information provided by the marker element 14 with corresponding information from an electronic medical record and thereby to check the correctness of the assignment. In this case, a notification may optionally be output to the user informing them that the correct ophthalmic implant 10 is used or a warning may be output to them informing them that the read-out identification and/or characterization information does not match the information provided in the medical record or elsewhere. Optionally, the identification and/or characterization information may be provided directly by the marker element 14. As an alternative or in addition, the information provided by the marker element 14 may specify a storage location at which the desired information is able to be retrieved, for example a server or network address. The system 30 may also be configured for example to carry out and/or suggest reordering of the ophthalmic implant 10 that is used in order to obtain a possible inventory.

FIG. 5 shows a system 42 for exchanging information with an ophthalmic implant 10 according to one exemplary embodiment. The system 42 is designed here as a diagnostic system and allows a patient's eye 34 to be diagnosed or measured. By way of example, the system 42 may have a wavefront aberrometer and/or an autorefractometer and/or a keratometer or be designed as such. In this case, the system 42 is also configured to enable an exchange of information with an ophthalmic implant of a patient's eye 34 to be diagnosed. For this purpose, the system 42 has a write and readout apparatus 44 that is designed to read out information for the machine-based identification and/or characterization of the ophthalmic implant 10 from a marker element 14 arranged in and/or on the ophthalmic implant 10 or to write such information to the marker element 14. The write and/or readout apparatus is in particular configured to optically detect the marker element 14 in order to read out and/or write the information and/or to contactlessly establish an electrical communication connection with the ophthalmic implant 10. A computing unit 38 connected to the write and readout apparatus 44 is designed to evaluate the read-out information and/or to provide the information to be written to the marker element 10. By way of example, the evaluation of the read-out information may comprise retrieving associated information from a separate information source, for instance from a server, which provides information for the ophthalmic implant identified by the read-out data. Information about the implantation process may also be provided by the marker element and/or a separate information source 46 (see FIG. 6), for example the time of implantation and any peculiarities and/or complications and/or information about past diagnoses. The system 42 furthermore comprises an output unit 40 in order to be able to output information to the user. By way of example, the output device 40 may have one or more displays in order to visually display information to the user. As an alternative or in addition, the output unit 40 may have one or more loudspeakers in order to output an acoustic signal. Optionally, the output unit 40 may also be designed to output a haptic signal.

FIG. 6 shows a schematic illustration of the interaction of a marker element 14 of an ophthalmic implant 10 with a write and/or readout apparatus 44 and a separate information source 46. In this case, the write and/or readout apparatus 44 constitutes an interface that makes it possible to retrieve information characterizing the ophthalmic implant 10 from the separate information source 46. To this end, for instance, the marker element 14 may provide information for the identification of the ophthalmic implant 10, such as a unique manufacturer number and/or identification number, which may be read out contactlessly by the write and/or readout apparatus 44 from the marker element 14. As an alternative or in addition, the marker element may provide one or more further items of information that may be used to retrieve the associated information from the separate data source 46, such as a network address and/or access data to the stored information. The write and/or readout apparatus 44 may in this case be integrated into another system, such as a diagnostic system and/or a surgical microscope. The system and in particular the write and/or readout apparatus 44 are in this case connected directly or indirectly, for example via a computing unit, to the separate information source 46. By way of example, the connection may be via a computer network, such as a local area network and/or the Internet. Optionally, the separate information source may be in the form of a data cloud that is provided for instance by the manufacturer of the system and/or the manufacturer of the ophthalmic implant and/or third parties. The separate information source 46 may also optionally be an electronic medical record, which is provided for instance locally in a clinic and/or a medical practice and/or is maintained by a health authority. Such exemplary embodiments offer the advantage of being able to provide information in the form of data both on the marker element 14 and on a separate information source 46. In particular, the separate information source 46 may be formed such that much larger amounts of data and information are able to be provided there than is possible on a marker element 14. A separate information source 46 may also offer the possibility of being able to adapt and/or supplement and/or expand and/or update the information stored there. In some exemplary embodiments, information stored on the marker element 14, in particular on a data storage unit 14b, may likewise be adapted and/or supplemented and/or expanded and/or updated.

FIG. 7 schematically shows, by way of example, a flowchart 100 of various optional use cases of a marker element 14 of an ophthalmic implant 10 over the lifetime of the ophthalmic implant, which provides information for the machine-based identification and/or characterization of the ophthalmic implant 10 and information for the machine-based determination of an alignment of the ophthalmic implant 10. The following use cases may be present here:

Use case 102: During the manufacture of the ophthalmic implant 10, the identification and/or characterization information may be used to uniquely identify the ophthalmic implant 10 and to rule out confusion. In addition, the information for the machine-based determination of an alignment of the ophthalmic implant 10 may be used to correctly position and/or orient the ophthalmic implant 10 during treatment.

Use case 104: During logistics, the identification and/or characterization information may be used to provide the ophthalmic implant 10 with the correct associated packaging and/or documentation and/or to supply the ophthalmic element 10 to the correct distribution channel. The risk of incorrect assignment of ophthalmic implant 10 and packaging and/or incorrect delivery to a non-intended recipient may thereby be reduced.

Use cases 106 to 110 may occur in particular in a clinic 200 and/or in a practice in which such ophthalmic implants are implanted.

Use case 106: Preoperatively, for instance, the information for the identification and/or characterization of the ophthalmic implant 10 may be used to achieve a correct assignment of the ophthalmic implant 10 to the intended patient's eye 34 and/or to check same. This makes it possible to reduce the risk of confusion or incorrect assignment, and the user is helped to check the correct assignment.

Use case 108: Intraoperatively, in particular, the information for the machine-based determination of the alignment of the ophthalmic implant 10 may be used to determine a relative alignment of the ophthalmic implant to the patient's eye in a machine-based manner, and thereby to offer the user or the surgeon assistance when positioning and/or orienting the ophthalmic implant 10. The identification and/or characterization information may also optionally be used to be able to check the correct assignment of the ophthalmic implant 10 and its properties to the patient's eye 34 again if necessary. Optionally, identification and/or characterization information may also be stored and/or modified on the marker element 14. By way of example, a time, such as a date of the implantation, may be stored on the marker element.

Use case 110: Postoperatively, the information may be used to check the implantation result after the implantation is complete. By way of example, the machine-based identification and/or characterization information may be used to check whether the intended ophthalmic implant 10 has been implanted. As an alternative or in addition, the position and/or orientation of the ophthalmic implant in the patient's eye may be checked based on the information for the identification and/or characterization of the ophthalmic implant 10, and possible deviations may be recognized.

Use case 112: During aftercare as well, for instance in regular examinations long after the implantation, the information may be used to identify and/or characterize the ophthalmic implant 10 and/or to check the alignment thereof. By way of example, the machine-based identification and/or characterization information may be used to check which ophthalmic implant 10 has been implanted and what properties this has. As an alternative or in addition, the position and/or orientation of the ophthalmic implant 10 in the patient's eye 34 may be checked based on the information for the identification and/or characterization of the ophthalmic implant 10, and possible deviations and/or changes over time may be recognized. Optionally, the information may also be used for possible complaints and/or quality control.

Further exemplary embodiments are described below.

According to one exemplary embodiment, the method and the system offer assistance with the centering of an IOL. In this case, the system uses an imaging method to recognize the actual position data (that is to say center, toric axis, physical edge) of the IOL based on the coded marker. As an alternative or in addition, the system uses a reader to read out the corresponding data contactlessly from the data storage unit, which is designed in this case so as to enable position detection. An electronic chip, contained in the data storage unit, of the marker element may alternatively also be detected visually with regard to its precise absolute position, provided that it is positioned in a corresponding region of the optical part of the IOL. In addition, the reader imports the IOL design specification (that is to say geometric dimensions, center, edge) from a separate information source, for example a server, or extracts it from the coded marker or the data storage unit.

### Based on

a) the reference data specifying the biometrics, geometry and properties of the eye and
b) the IOL design specification and the actual IOL position data,
the system generates an electronic output of information for the user in order to indicate the correct centering of an IOL. The system gives the user direct feedback, for example in visual form and/or in auditory form.

One special feature is that the data of the coded marker and/or of the data storage unit provide the physical position of the IOL edge and of the IOL center within the operation scene and at least offer error recognition that ensures that the system correctly recognizes the IOL center.

According to a further exemplary embodiment, the method and the system offer assistance with the tilt positioning or alignment of an IOL.

In this case, the system preferably uses an imaging method to recognize the actual position data (that is to say center, physical edge) of the IOL based on the coded marker. In addition, it imports the IOL design specification (that is to say geometric dimensions, center, edge) from a separate information source, for instance a server, or extracts it from the coded marker. Based on
a) the IOL design specification and the actual IOL position data,
b) the optical parameters from the observation using the microscope and/or the camera,
c) optionally: from preoperative reference data or intraoperative measurement data specifying the biometrics and geometry of the patient's eye (for example reference coordinate system),

the system generates an electronic output in order to control the correct setting of a tilt or inclination of the intraocular lens. The system gives the user direct feedback, for example in visual form and/or in auditory form. One special feature is that the data of the coded marker provide the physical position of the IOL within the operation scene and at least offer error recognition that ensures that the system correctly recognizes the IOL.

As an alternative or in addition, the system uses a reader to read out the corresponding data from the data storage unit, which is designed in this case so as to enable position detection. An electronic chip contained in the data storage unit may alternatively also be detected visually with regard to its precise absolute position, provided that it is positioned in a corresponding region of the optical part of the IOL.

According to a further exemplary embodiment, the system and the method offer assistance with the depth positioning of an IOL.

The system uses an imaging method to recognize the actual position data (that is to say center, physical edge, unique visual identification pattern) of the IOL based on the coded marker. In addition, it imports the IOL design specification (that is to say geometric dimensions, center, edge) from a separate information source, for example a server, and/or extracts it from the coded marker.

### Based on

a) the reference data specifying the biometrics, geometry and properties of the patient's eye;
b) the IOL design specification and the actual IOL position data;
c) the optical parameters from the observation of the patient's eye and of the ophthalmic implant using a microscope and/or a camera,

the system generates an electronic output of information for the user that provides depth information about the IOL.

As an alternative or in addition, the system uses a reader to read out the corresponding data contactlessly from the data storage unit, which is designed in this case so as to enable position detection. An electronic chip contained in the data storage unit may alternatively also be detected visually with regard to its precise absolute position.

According to one exemplary embodiment, the system and the method offer for setting the imaging focus and/or zoom control.

The system in this case uses an imaging method to recognize the actual position data (that is to say center, physical edge, toric axis, unique visual identification pattern) of the IOL based on the coded marker. In addition, it imports the IOL design specification (that is to say geometric dimensions, center, edge) from another information source, for example a server, and/or extracts it from the coded marker.

### Based on

a) the knowledge of the structure and content of the coded marker,
b) the results of image recognition or detection of the coded marker,
c) the optical parameters from the observation using the microscope and/or the camera, the system is able to control or set the image focus and/or zoom so as to display the implanted IOL as best possible.

A description is given below of a system and a method for analyzing the IOL position after a cataract operation based on coded IOL markers according to one exemplary embodiment.

Provision is made for a system for recognizing and identifying coded IOL markers or a data storage unit in medical diagnostic devices. A description is also given of methods for performing data processing, visualizing and identifying the IOL alignment on the basis of coded IOL markers or a data storage unit.

### The System Comprises:

a) an ophthalmological implant (for example an IOL) having a marker element, in particular a coded marker and/or a data storage unit;
b) an optical readout apparatus for reading out the information from the marker element. The readout apparatus may be designed for instance as a medical diagnostic device (for example slit lamp, biometric device, autorefractor, retroillumination). As an alternative or in addition, the system may use a separate reader to read out the corresponding data contactlessly from the data storage unit.
c) optionally, an image acquisition device, in particular a camera system;
d) application software for (i) performing data processing and visualizing the information provided by the marker element, and (ii) recognizing the position of the ophthalmic implant on the basis of the information provided by the marker element;
e) optionally: an IOL database containing an implant specification, which may form part of the system or may be provided separately from the system, for instance on a server able to be reached via a network;
f) optionally: a connected electronic medical record (EMR) containing information about the patient and/or the operation results, wherein the electronic medical record optionally allows results of the position analysis performed by the system to be stored.

The system is designed in this case such that it enables the following functionalities:
a) reading out and visualizing at least some of the information provided by the marker element;
b) optionally: assisting the surgeon with analysis of the toric IOL alignment, including changes over time;
c) optionally: assisting the surgeon with the analysis of the
   i) centering of an IOL;
   ii) tilt or inclination positioning or alignment of an IOL;
   iii) depth positioning of an IOL;
d) optionally: analyzing changes in IOL positioning or alignment over time (for example across the healing process).

The postoperative option to read out and visualize the data provided by the marker element, also referred to as IOL data within the scope of the disclosure, enables the surgeon to check and/or confirm that the correct intraocular lens (IOL) has been implanted. It may also be used as a legal argument in the event of a liability claim, for instance to provide evidence that the intended ophthalmic implant has been correctly implanted or that an incorrect implant has been implanted and/or that an otherwise defective implantation has taken place. The assistants for assisting the torics, centering, tilting and depth positioning of an IOL are designed to allow the surgeon to efficiently assess and/or evaluate the postoperative result of the IOL implantation. The surgeon may also use these assistants to track the results, for example the rotational stability of toric IOLs in individual patients over time. In addition, the assistants may be used while the surgeon is postoperatively adjusting the IOL alignment if additional IOL alignment is required.

The functionalities are described in detail below.
a) Reading out and visualizing the IOL data

The system uses an imaging method to recognize the presence of the marker element and
a) to extract the IOL design specification (that is to say geometric dimensions, center, edge) from the marker element and/or import it from a database;
b) to apply error recognition/correction to the coded data;
c) to visualize the IOL data in textual and/or graphical form for the user or surgeon.

The system provides the user with information for the identification and/or characterization of the implanted intraocular lens, which information may be used to check and/or confirm that the correct IOL has been implanted, or to specify the implant. As an alternative, the system uses a reader to read out the corresponding data from a data storage unit of the marker element.
b) Assistant for analyzing the toric IOL alignment, including changes over time

The system uses an imaging method to extract the toric axis of the IOL, which may be derived from the marker element. Based on the
a) reference data specifying the biometrics, geometry and properties of the eye;
b) the toric axis of the IOL; and/or
c) an operation plan/report,

the system generates an electronic output to provide the user with a degree of toric IOL axis misalignment (delta of the actual IOL alignment compared to a reference IOL alignment). Optionally, the analysis results may be documented in the EMR.

As an alternative, the system uses a reader to read out the corresponding data contactlessly from a data storage unit of the marker element, which data storage unit is designed in this case so as to enable position detection. An electronic chip contained in the data storage unit may alternatively also be detected visually with regard to its precise absolute position and used to ascertain the position and/or alignment of the ophthalmic implant.
c) Assistant for analyzing the centering of an IOL

The system uses an imaging method to recognize the actual position data (that is to say center, physical edge, toric axis, unique visual identification pattern) of the IOL based on the marker element. In addition, it imports the IOL design specification (that is to say geometric dimensions, center, edge) or extracts it from the marker element.

### Based on the

a) reference data specifying the biometrics, geometry and properties of the eye;
b) the IOL design specification;
c) the actual IOL position data derived from the coded marker;

the system generates a, generally electronic, output to provide the user with a degree of IOL decentering, that is to say a degree of deviation of the actual position and/or alignment from an intended position and/or alignment of the IOL relative to the patient's eye. Optionally, the analysis results may be documented in the EMR.

As an alternative, the system uses a reader to read out the corresponding data contactlessly from the data storage unit of the marker element, which data storage unit is designed in this case so as to enable position detection. An electronic chip contained in the data storage unit may alternatively also be detected visually with regard to its precise absolute position.
d) Assistant for analyzing the tilt alignment of an IOL

The system uses an imaging method to recognize the actual position data and/or the alignment (that is to say center, physical edge, toric axis, unique visual identification pattern) of the IOL based on the marker element, in particular a coded marker. In addition, it imports the IOL design specification (that is to say geometric dimensions, center, edge) or extracts it from the coded marker.

### Based on the

a) IOL design specification;
b) the actual IOL position data derived from the coded marker;
c) optical parameters from the observation using the diagnostic device and/or the camera; and
d) optionally: preoperative reference data, intraoperative measurement data and/or postoperative measurement data specifying the biometrics and geometry of the eye (for example reference coordinate system), the system generates an optional electronic output to provide the user with a degree of IOL inclination or tilt. Optionally, the analysis results may be documented in the EMR.

As an alternative, the system uses a reader to read out the corresponding data contactlessly from the data storage unit, which is designed in this case so as to enable position detection. An electronic chip contained in the data storage unit of the marker element may alternatively also be detected visually with regard to its precise absolute position.
e) Assistant for analyzing the depth positioning of an IOL

The system uses an imaging method to recognize the actual position data (that is to say center, physical edge, toric axis, unique visual identification pattern) of the IOL based on the coded marker. In addition, it imports the IOL design specification (that is to say geometric dimensions, center, edge) from a separate information source, for instance a server, or extracts it from the coded marker.

### Based on the

a) reference data specifying the biometrics, geometry and properties of the eye;
b) the IOL design specifications;
c) actual IOL position data derived from the coded marker,
d) optical parameters from the observation using the diagnostic device and/or the camera, that is to say an image acquisition device,

the system generates an electronic output in order to provide the user with depth information about the IOL. Optionally, the analysis results may be documented in the EMR.

As an alternative, the system uses a reader to read out the corresponding data contactlessly from the data storage unit, which is designed in this case so as to enable position detection. An electronic chip contained in the data storage unit may alternatively also be detected visually with regard to its precise absolute position.

Further optional and where applicable additional workflow use cases are described below.

The marker element may have a coded marker and/or a data storage unit. The coded marker and/or the data storage unit (electronic tag) on the IOL optionally enable/enables full traceability of the implant throughout the entire life of the implant. If necessary, the coded marker or the data storage unit may be read by a system, as described in the disclosure, in order to identify the implant and/or to obtain individual implant specifications. It should be noted here that the system is optionally adapted to standard devices and modules used for the respective application. In principle, the described systems are based on the marker element of the ophthalmic implant, in particular a coded IOL marker and/or a data storage unit, a device having imaging capabilities and/or an electronic reader for reading the data/information, stored in the coded marker or in the data storage unit, in relation to the relevant intraocular lens, and a computer for extracting and processing the information.
a) During the manufacture of the intraocular lens or of the ophthalmological implant

When the coded marker or the data storage unit is placed inside the IOL material, the generation of the intraocular lens may be tracked throughout the entire manufacturing process, including turning, milling, sterilization and packaging. As an alternative or in addition, a marker element 14 may however also be applied at the end of the process of manufacturing the ophthalmic implant 10. In one optional embodiment, the markers may be used to align the samples in production in order to ensure that toricity and haptic elements lie in the correct axis. As an alternative or in addition, a data storage unit may be used for the alignment. In addition to the variants described above with the formation of special antennas able to be detected in terms of their spatial position or by using multiple subunits of the data storage units to determine the position, a data storage unit in the form of an electronic chip may also be made visually visible and may be used as a reference structure. The traceability of the individual intraocular lens or ophthalmological implants during production makes it possible to manufacture individual IOLs or implants, instead of manufacturing larger quantities of a specification. It is also possible to automate production. Since the implant specification is stored on the coded marker or in the data storage unit, more accurate specifications, specifying for example the exact dioptric strength, are also possible.
b) For quality control of the intraocular lens or ophthalmological implant

A comparison between coded marker or data storage unit on the IOL and the pre-existing Unique Device Identifier (UDI) on the lens packaging enables a further safety inspection at the end of the production process. Here, the system is able to extract the lens information provided by the coded marker or the data storage unit on the IOL and the lens information provided by the UDI, to compare said lens information and to generate an electronic output that indicates that the information either matches or does not match. When using a coded marker, it is necessary to provide access to the packaging (for example a transparent window) in order to have visual contact with the coded marker on the implant. When using a data storage unit, such a window is not necessary.
c) Postoperative IOL adjustments

The system for analyzing the IOL position postoperatively or after a cataract operation is combined with postoperative IOL adjustment methods (for example thermal, electroactive, magnetic, light-induced, acoustic). This enables assistance functions that assist the surgeon or enable automatic IOL adjustment. Both result in higher precision in postoperative IOL adjustment.
d) Spectacle lenses optimized for the IOL

A coded marker or a data storage unit on the IOL may be read by an optician using a device. The optician receives information about the IOL implanted in the patient and is able to incorporate the IOL design specification in the eyeglass lens calculation.
e) Digital IOL pass

The coded marker or the data storage unit on the IOL may provide the information specified on the implant pass in digital form. The patient therefore always carries a digital copy.
f) Legal argument

The coded marker or the data storage unit on the IOL provides the implant specification and may be used as a legal argument in the event of liability claims.
g) Automated patient identification on devices

By linking the coded marker or the data storage unit on the IOL to patient information (for example from the EMR system), the system enables automated identification of patients on clinical devices. Instead of first having to register each patient on the respective devices, staff are able to start working with the devices immediately.
h) Manufacturer complaint/quality management

Coded markers or data storage units on and/or in intraocular lenses are able to be read with the described system in the implanted or explanted state in order to identify and track the implant. This helps to understand customer complaints and to identify the source of the complaint.
i) Diagnostic measurements

Various diagnostic measuring devices are able to read out the implant, extract the implant specification, for example material properties and optical design, and incorporate the implant specifications into their measuring methods. This makes it possible to provide more reliable measurements and more precise measurement results.
j) Analysis of the operation result and optimization of the IOL constant

Devices for measuring the operation result, such as for example autorefractors or biometrics devices, are able to identify the implant, extract the implant specification, combine the results of the measurement with the implant specification and upload them to a software application for results analysis and for IOL constant optimization.
k) Tracking of eye movements

In upcoming eye measurements or eye operations, the devices are able to recognize the actual position (that is to say center, physical edge, toric axis, unique visual identification pattern) of the IOL based on the marker element, in particular a coded marker. This recognition makes it possible to track eye movements, which may be incorporated into
(i) measurement methods of diagnostic devices in order to reduce measurement errors
(ii) for assistance functions during the operation in order to assist the performance of the surgeon, for example automatic adjustments to the surgical microscope or imaging settings.

As an alternative, the corresponding data are read out contactlessly from the data storage unit, which is designed in this case so as to enable position detection. An electronic chip contained in the data storage unit may again alternatively also be detected visually with regard to its precise absolute position.

### LIST OF REFERENCE SIGNS

10 ophthalmic implant
12 intraocular lens
12a optical part
12b non-optical part
12c haptic element
14 marker element
14a coded marker
14b data storage unit
16 chip
18 antenna structure
20 machine-readable code
30 system for providing machine-based assistance for a user when implanting an ophthalmic implant
32 surgical microscope
34 patient's eye
36 patient table
38 computing unit
40 output unit
42 system for exchanging information with an ophthalmic implant
44 write and/or readout apparatus
46 separate information source
100 flowchart of the life cycle of an ophthalmic implant
102-112 use cases of the marker element
200 clinical use cases
1000 visualization of the toric axis of an IOL
1002 rotation angle indicators
1004 visualization of the astigmatism axis of the patient's eye

1-31. (canceled)

32. A method for machine-based determination of an alignment of an ophthalmic implant having at least one marker element relative to a patient's eye for assisting an implantation operation, the method comprising:
reading out information for at least one of machine-based identification and characterization of the ophthalmic implant from the marker element;
machine-based detection of the marker element;
machine-based determination of at least one of a position and an alignment of the marker element;
machine-based determination of at least one of a position and an alignment of at least part of the patient's eye; and
intraoperatively determining the alignment of the ophthalmic implant relative to the patient's eye based on the determined position and/or alignment of the marker element and the determined position and/or alignment of the at least part of the patient's eye.

33. The method as claimed in claim 32, further comprising preoperatively and/or intraoperatively assigning or checking of an assignment of the ophthalmic implant to a patient's eye based on the information read out from the marker element.

34. The method as claimed in claim 32, wherein the ophthalmic implant comprises or consists of an intraocular lens (IOL) and wherein the method further comprises at least one of the following steps based on the information read out from the marker element:
visualizing IOL data;
documenting IOL data; and
checking that an actual IOL matches the IOL intended for implantation;
confirming that the actual IOL matches the IOL intended for implantation;
confirming the actual IOL and comparing the actual IOL with the IOL intended for the implantation;
comparing the information read out from the marker element with corresponding information from an electronic medical record;
providing operation planning data containing IOL identification data based on the information read out from the marker element; and
exporting data from the ophthalmic implant to an external computer system for computing and processing data involving the information read out from the marker element.

35. The method as claimed in claim 32, wherein the ophthalmic implant comprises or consists of an intraocular lens, IOL, and wherein based on the information read out from the marker element at least one of the following information is provided:
IOL design specification data;
IOL geometry data;
IOL position data; and
a physical position of an IOL edge and of an IOL center within an operation scene;
wherein the method further comprises at least one of the following steps using at least one of the IOL design specification data, the IOL geometry data, and the IOL position data;
assisting a centering of the IOL;
assisting a tilting or inclination positioning of the IOL;
assisting a depth positioning of the IOL along the optical axis of the patient's eye;
assisting for setting at least one of an imaging focus and a zoom control of an image acquisition system used during positioning the IOL; and
carrying out an error recognition to ensure a correct recognition of the IOL center.

36. Method as claimed in claim 32, wherein the machine-based determination of at least one of a position and an alignment of at least part of the patient's eye is carried out at least partly using OCT imaging.

37. The method as claimed in claim 32, wherein the method comprises outputting, optionally in visual form and/or in auditory form, information about the position and/or the alignment of the ophthalmic implant relative to the patient's eye during and/or after an operation of implanting the ophthalmic implant.

38. The method as claimed in claim 32, wherein the determination of the alignment of the ophthalmic implant relative to the patient's eye is repeated multiple times and optionally carried out continuously.

39. The method as claimed in claim 32, wherein the marker element has a marking in the form of a machine-readable code or consists thereof, and
wherein the readout of the information for the machine-based identification and/or characterization of the ophthalmic implant from the marker element comprises reading out the information for the machine-based identification and/or characterization of the ophthalmic implant from the machine-readable code.

40. The method as claimed in claim 39, wherein the machine-readable code has at least one of a one-dimensional barcode, a two-dimensional matrix code, a dot matrix containing marking dots having a pseudo-random irregular character, and a three-dimensional matrix code or consists thereof.

41. The method as claimed in claim 32, wherein the marker element comprises a data storage unit or consists thereof, and
wherein the readout of information for at least one of the machine-based identification and characterization of the ophthalmic implant comprises electronic readout of data from the data storage unit including the information for at least one of the machine-based identification and characterization of the ophthalmic implant.

42. The method as claimed in claim 41, wherein the machine-based determination of at least one of the position and alignment of the marker element comprises determination of at least one of a position and alignment of the data storage unit.

43. A method for the postoperative machine-based determination of an alignment of an ophthalmic implant having at least one marker element relative to a patient's eye, the method comprising:
machine-based detection of the marker element;
machine-based determination of at least one of a position and an alignment of the marker element;
machine-based determination of at least one of a position and an alignment of at least part of the patient's eye;
determining the alignment of the ophthalmic implant relative to the patient's eye based on at) least one of the determined position and alignment of the marker element and at least one of the determined position and alignment of the at least part of the patient's eye; and
reading out information for the machine-based identification of the ophthalmic implant from the marker element.

44. The method as claimed in claim 43, further comprising at least one of the following steps:
postoperatively checking of an assignment of the ophthalmic implant to the patient's eye in which the ophthalmic implant is implanted based on the information for the machine-based identification of the ophthalmic implant read out from the marker element; and
postoperatively providing information for at least one of a quality control and a complaint based on the information read out from the marker element;
carrying out a postoperative adaptation of the ophthalmic implant using a postoperative alignment method;
postoperatively providing an IOL design specification data from the marker element to an optician for incorporating the IOL design specification data in an eyeglass lens calculation;
providing information specified on an implant pass in digital form based on the information read out from the marker element; and
postoperatively identifying a patient having the ophthalmic implant implanted in one of the patient's eye based on the information read out from the marker element.

45. The method as claimed in claim 44, wherein the marker element has a marking in the form of a machine-readable code or consists thereof, and
wherein the readout of the information for the machine-based identification and/or characterization of the ophthalmic implant from the marker element comprises reading out the information for the machine-based identification and/or characterization of the ophthalmic implant from the machine-readable code.

46. The method as claimed in claim 45, wherein the machine-readable code has at least one of a one-dimensional barcode, a two-dimensional matrix code, a dot matrix containing marking dots having a pseudo-random irregular character, and a three-dimensional matrix code or consists thereof.

47. The method as claimed in claim 46, wherein the marker element comprises a data storage unit or consists thereof, and
wherein the readout of information for at least one of the machine-based identification and characterization of the ophthalmic implant comprises electronic readout of data from the data storage unit including the information for at least one of the machine-based identification and characterization of the ophthalmic implant.

48. The method as claimed in claim 47, wherein the machine-based determination of at least one of the position and alignment of the marker element comprises determination of at least one of a position and an alignment of the data storage unit.

49. A method for manufacturing and packaging an intraocular lens, IOL, comprising:
providing an IOL having at least one optical part and at least one haptic element connected to the optical part;
arranging a marker element containing information for at least one of the machine-based identification and characterization of the IOL at least partially in or on at least one of the optical part and the at least one haptic element such that, in a state in which the IOL is implanted in a patient's eye, the information for the identification and/or characterization of the IOL is able to be read out in a machine-based manner and information for the machine-based determination of the alignment of the IOL is able to be acquired optically based on the marker element; and
packaging the intraocular lens and using the information for the machine-based identification contained in the marker element for identifying the intraocular lens during the packaging of the intraocular lens.

50. The method as claimed in claim 49, wherein arranging the marker element comprises affixing a machine-readable code, wherein the machine-readable code has at least one a one-dimensional barcode, a two-dimensional matrix code, a dot matrix containing marking dots having a pseudo-random irregular character, and a three-dimensional matrix code or consists thereof.

51. The method as claimed in claim 49, wherein arranging the marker element comprises or consists in installing a data storage unit that contains at least one of the information for at last one of the machine-based identification and characterization of the IOL and the information for the machine-based determination of the alignment of the IOL in the form of electronically readable data.

52. A method for checking an ophthalmic implant comprising or consisting of an intraocular lens (IOL) and having at least one marker element, the method comprising:
reading out information for at least one of machine-based identification and characterization of the ophthalmic implant from the marker element,
wherein the method further comprises at least one of the following steps based on the information read out from the marker element;
preoperatively and/or intraoperatively assigning or checking of an assignment of the ophthalmic implant to a patient's eye;
visualizing IOL data;
documenting IOL data;
checking that the actual IOL matches the IOL intended for implantation;
confirming that the actual IOL matches the IOL intended for implantation;
confirming the actual IOL and comparing it with the IOL intended for the implantation;
comparing the information read out from the marker element with corresponding information from an electronic medical record;
providing operation planning data containing IOL identification data based on the information read out from the marker element; and
exporting data from the ophthalmic implant to an external computer system for computing and processing data involving the information read out from the marker element.

53. The method as claimed in claim 52, wherein based on the information read out from the marker element at least one of the following information is provided:
IOL design specification data;
IOL geometry data;
IOL position data; and
a physical position of an IOL edge and of an IOL center within an operation scene;
wherein the method further comprises at least one of the following steps using at least one of the IOL design specification data, the IOL geometry data, and the IOL position data;
assisting a centering of the IOL;
assisting a tilting or inclination positioning of the IOL;
assisting a depth positioning of the IOL along the optical axis of the patient's eye;
assisting for setting at least one of an imaging focus and a zoom control of an image acquisition system used during positioning the IOL; and
carrying out an error recognition to ensure a correct recognition of the IOL center.
